(19) United States
(12) Patent Application Publication
(10) Pub. No.: US 2002/0156466 A1
(43) Pub. Date: Oct. 24, 2002

(54) Surgical system

(75) Inventors: Tomohisa Sakurai, Sagamihara-shi (JP); Yoshitaka Honda, Tokorozawa-shi (JP); Kazue Tanaka, Sagamihara-shi (JP)

Correspondence Address: Paul J. Esatto, Jr., Scully, Scott, Murphy & Presser, 400 Garden City Plaza, Garden City, NY, 11530, US

(73) Assignee: OLYMPUS OPTICAL CO., LTD., TOKYO (JP)

(21) Appl. No.: 10/123,783

(22) Filed: Apr. 16, 2002

(30) Foreign Application Priority Data
Apr. 18, 2001 (JP) 2001-120238

(51) Int. Cl. A61B 17/00; A61B 17/32

(52) U.S. Cl. 606/1; 606/169

(57) ABSTRACT

A surgical system is capable of easily selecting one of treating tools used in surgical treatment by operating a push button switch provided on a remote controller disposed near a corresponding treating tool. When a remote controller is operated, an ID signal uniquely assigned to the remote controller is transmitted using a radio wave to a foot switch controller. An ID discriminator discriminates the ID transmitted from the remote controller thereby determining which remote controller has been selected. A selection acknowledgement signal is returned to the operated remote controller. In response, an LED disposed on the operated remote controller is lit so that a human operator can easily recognize that the remote controller has been selected.

This application claims benefit of Japanese Application No. 2001-120238, filed in Japan on Apr. 18, 2001, the content of which are incorporated by this reference.

### BACKGROUND OF THE INVENTION

1. Field of the Invention

The present invention relates to a surgical system capable of making treatment using two or more different types of energy such as high-frequency electric power and ultrasonic vibration.

2. Description of the Related Art

Great advances in the medical science and improvements in surgical equipment have been made, and now surgery can be performed using various kinds of advanced surgical equipment. For example, an ultrasonic surgical apparatus using ultrasonic vibration has been developed for use instead of an electric cautery. For more detailed information on the ultrasonic surgical apparatus, reference is made to Japanese Unexamined Patent Application Publication No. 9-38098. Such an ultrasonic surgical apparatus and an electric cautery are effectively used in practical surgery. Those apparatuses are provided in the form of separate units that are controlled by separate control switches (such as foot switches).

It has been proposed to construct a surgical system such that one of apparatuses can be selected and the selected apparatus can be controlled by a single foot switch provided for common use, wherein an operation performed when the foot switch is pressed can be selected by a human operator via a switch disposed on a control panel or via a remote switch that can be directly operated by the human operator or via a voice control system. For more detailed information, refer to Japanese Patent Application No. 10-136645.

A system has also been developed in which an ultrasonic surgical apparatus and an electric cautery apparatus are combined. An ultrasonic emulsification/aspiration apparatus is capable of emulsifying and aspirating substantially only an organ part so that elastic tissue such as a blood vessel or nervous tissue remains without being destroyed. Even in this case, fine blood vessels can be destroyed and bleeding occurs. Thus, in this system, the electric cautery incorporated therein is used to stanch the bleeding thereby allowing the surgery to be continuously performed. Japanese Unexamined Patent Application Publication No. 5-23348 discloses a system in which ultrasonic power and high-frequency electric power can be output alternately or simultaneously.

However, the conventional techniques have the following problems, which result in bad operability.

That is, in the conventional technique, a desired apparatus is selected by operating a switch disposed on a front panel located in an unclean region, and thus a surgeon cannot directly operate the switch during a surgical operation. When it is needed to operate the switch, the switch is operated by another person such as a nurse or the like. This results in inefficiency and complexity.

Although a desired apparatus can be directly selected by the surgeon using a remote switch, it is impossible to easily recognize which button corresponds to which apparatus or hand piece, and the provision of the remote switch results in an increase in the number of cables disposed in an operating room, and thus a troublesome problem can occur.

When the surgeon confirms whether a desired apparatus has been correctly selected by a selector on the basis of an indication on a panel of the selector, there is a possibility that the indication cannot be easily viewed depending on the location of the selector. This makes it difficult to confirm whether a desired apparatus has been correctly selected.

In a conventional surgical system in which an ultrasonic surgical apparatus and an electric cautery are combined, the system is designed only for applications of the ultrasonic aspiration, and thus energy is output only alternately or simultaneously from the two apparatuses in a similar manner as is output from individual apparatuses. This makes it difficult to use output energy in an efficient manner.

### SUMMARY OF THE INVENTION

It is an object of the present invention to provide a surgical system capable of easily selecting one of apparatuses controllable by an output switch and capable of making treatment in a more effective manner using a combination of an electric cautery apparatus and an ultrasonic surgical apparatus.

According to an aspect of the present invention, there is provided a surgical system comprising: a first treating apparatus for generating first treating energy; a first treating tool for treating a patient's body part on the basis of the first treating energy generated by the first treating apparatus; a first setting switch disposed in the vicinity of the first treating tool, for making setting such that the first treating energy is used in treatment; a second treating apparatus for generating second treating energy; a second treating tool for treating a patient's body part on the basis of the second treating energy generated by the second treating apparatus; a second setting switch disposed in the vicinity of the second treating tool, for making setting such that the second treating energy is used in treatment; a display for indicating the states of the first and second setting switches; a foot switch for issuing a treatment start command; and a controller for controlling the first and second treating apparatuses in accordance with the states of the first and second setting switches and the foot switch. In this system constructed in the above-described manner, the first or second treating tools can be easily set into an enabled state by operating the first or second setting switch disposed near the first and second treating tools, and it is possible to easily conform the setting states of the respective setting switches on the basis of the indication of the display. And after confirming the states, treatment can be started by the foot switch.

These object and advantages of the present invention will become further apparent from the following detailed explanation.

### BRIEF DESCRIPTION OF THE DRAWINGS

FIG. 1 is a diagram showing a surgical system according to a first embodiment of the present invention;

FIG. 2 is a block diagram showing the internal structures of a remote controller and a foot switch controller;

FIG. 3 is a diagram showing an example of means for attaching a remote controller;

FIG. 4 is a block diagram showing a foot switch control circuit disposed in a foot switch unit;

FIG. 5 is a flow chart of a presetting process;

FIG. 6A is a timing chart showing a foot switch signal that is output when a foot switch (pedal A) is pressed in a mixed output mode;

FIG. 6B is a timing chart showing high-frequency cutting power that is output when the foot switch (pedal A) is pressed in the mixed output mode;

FIG. 6C is a timing chart showing 100% ultrasonic power that is output when the foot switch (pedal A) is pressed in the mixed output mode;

FIG. 7A is a timing chart showing a foot switch signal that is output when a foot-switch (pedal B) is pressed in the mixed output mode;

FIG. 7B is a timing chart showing high-frequency cutting power that is output when the foot switch (pedal B) is pressed in the mixed output mode;

FIG. 7C is a timing chart showing 70% ultrasonic power that is output when the foot switch (pedal B) is pressed in the mixed output mode;

FIG. 8 is a circuit diagram of a foot switch control circuit of a surgical system according to a second embodiment of the present, invention; and

FIG. 9 is a diagram showing a modification of a foot switch unit in which a foot switch controller is disposed.

### DESCRIPTION OF THE PREFERRED EMBODIMENTS

The present invention is described in further detail below with reference to preferred embodiments in conjunction with the accompanying drawings.

First Embodiment

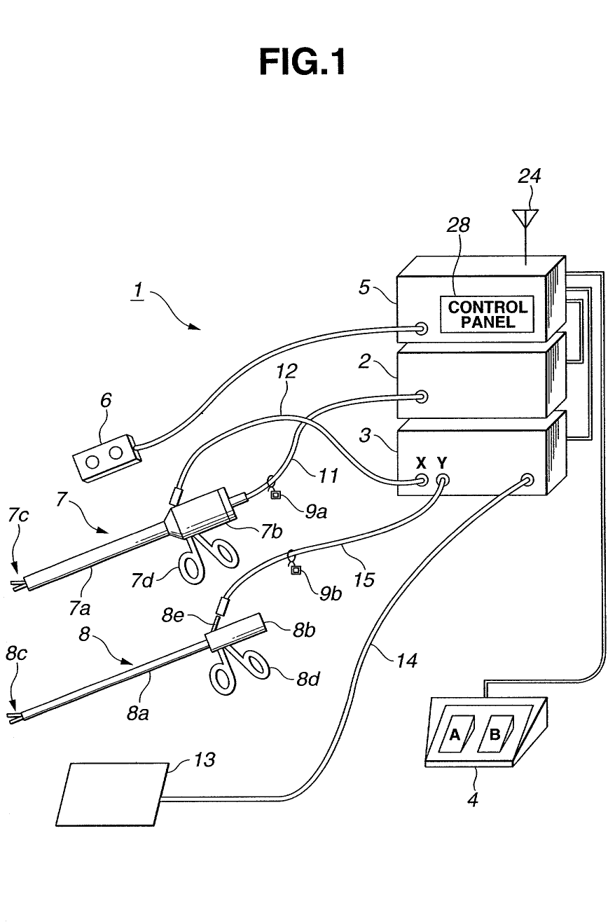

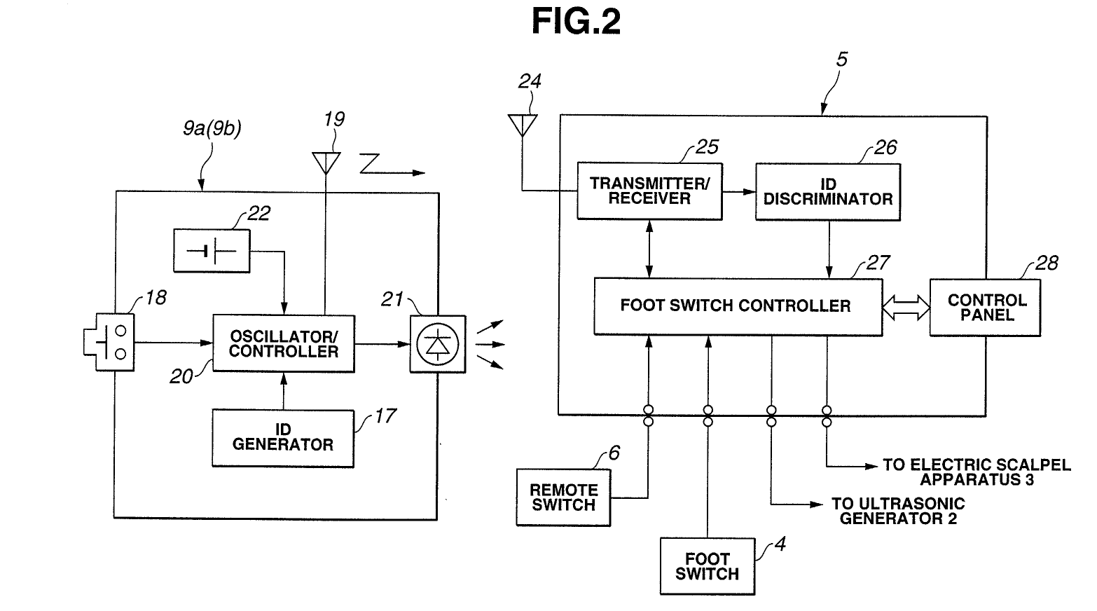

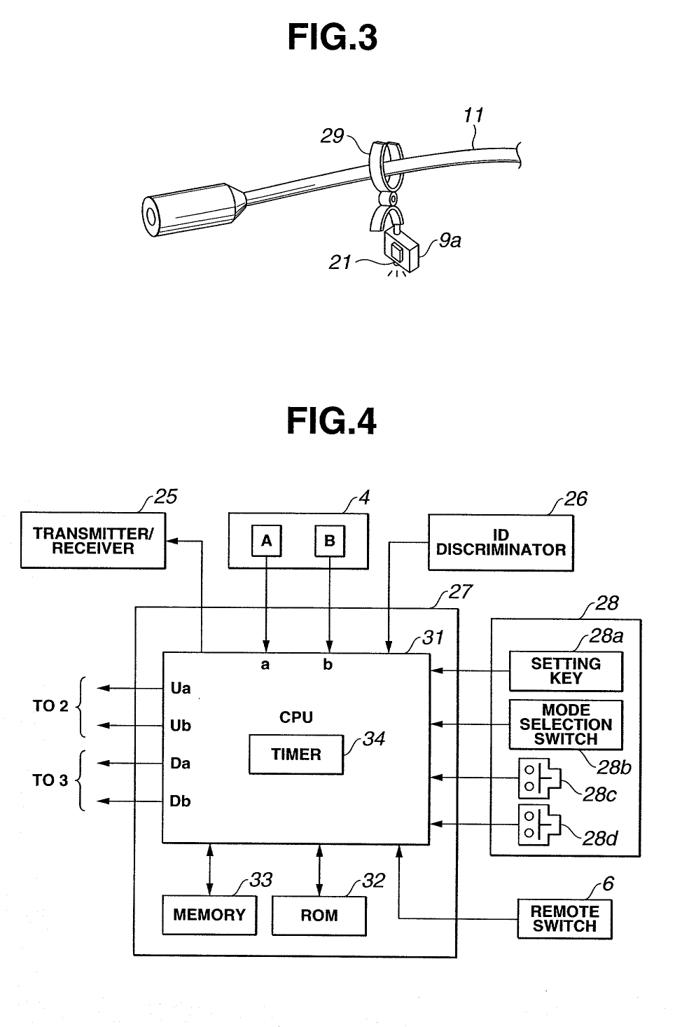

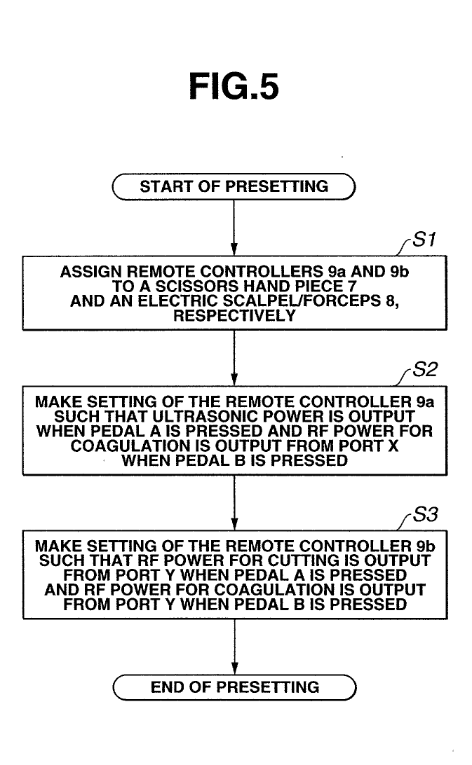

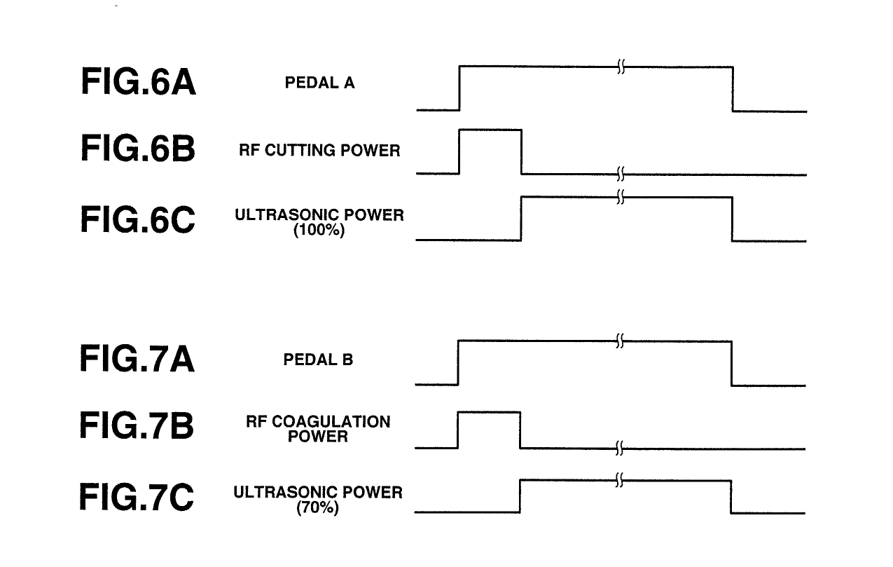

A first embodiment of the present invention is described below with reference to FIGS. 1 to 7, wherein FIG. 1 shows a surgical system according to the first embodiment; FIG. 2 shows the internal structures of a remote controller and a foot switch controller; FIG. 3 shows means of attaching the remote controller; FIG. 4 shows the foot switch control circuit disposed in the foot switch unit; FIG. 5 shows a presetting process; FIGS. 6A to 6C and FIGS. 7A to 7C are timing charts showing signals/power output from blocks in response to an operation of the foot switch in the mixed output mode, and more specifically, FIG. 6A shows a signal that is output from the foot switch when a pedal A is pressed; FIG. 6B shows high-frequency cutting power that is output when the pedal A is pressed; FIG. 6C shows 100% ultrasonic power that is output when the pedal A is pressed; FIG. 7A shows a signal that is output when a pedal B is pressed; FIG. 7B shows high-frequency cutting power that is output when the pedal B is pressed; and FIG. 7C shows 70% ultrasonic power that is output when the pedal B is pressed.

As shown in FIG. 1, the surgical system 1 according to th first embodiment of the present invention includes a plurality of apparatuses for generating treating energy and a plurality of treating means for making treatment using the treating energy.

More specifically, the surgical system 1 includes an ultrasonic generator 2 for generating ultrasonic energy; a high-frequency electric cautery apparatus 3 for generating high-frequency energy (hereinafter referred to as an electric cautery apparatus); a foot switch 4 for selectively issuing a treatment start command to the ultrasonic generator 2 or the electric cautery apparatus 3; a foot switch controller 5 for selectively transferring an operation signal output from the foot switch 4 to the ultrasonic generator 2 or the electric cautery apparatus 3; a remote switch 6 for remote control, connected to the foot switch controller 5; and two treating tools having different functions, and more particularly, a scissors hand piece 7 having a capability of performing cutting and coagulating using ultrasonic vibration and also capable of performing stanching using a high-frequency electric current and a high-frequency electric cautery/hemostat (also referred to as an electric cautery/hemostat) 8 having a capability of performing stanching using a high-frequency electric current.

Wireless remote control switches (hereafter, referred to as remote controllers) 9a and 9b for making setting of the respective treating tools such as the scissors hand piece 7 and the electric cautery/hemostat 8 are disposed near the treating tools.

The scissors hand piece 7 includes an ultrasonic vibrator (not shown) disposed in an operating part 7b on the back end of an elongated sheath 7a, wherein the ultrasonic vibrator is connected to the ultrasonic generator 2 via a cable 11.

When the scissors hand piece 7 is in a standby state that can be achieved by operating the remote controller 9a, if the pedal A of the foot switch unit 4 is pressed, an operation signal is transmitted to the ultrasonic generator 2 via the foot switch controller 5. In response, the ultrasonic generator 2 outputs ultrasonic energy to the ultrasonic vibrator, whereby ultrasonic vibration occurs in the ultrasonic vibrator. The ultrasonic vibration is transmitted to a treating part 7c disposed on the front end of the sheath 7a via an ultrasonic transmission rod in the sheath 7a, and the ultrasonic vibration is applied to a patient's body part such as tissue nipped by the treating part 7c thereby performing cutting or coagulating. Handles 7d for controlling the opening/closing of pivoting portions of the treating part 7c are provided on the operating part 7b.

The scissors hand piece 7 is also connected via an (active) cable 12 to one output port X of the electric cautery apparatus 3. When the scissors hand piece 7 is in the standby state in which the scissors hand piece 7 is ready for use, if the other pedal B of the foot switch unit 4 is pressed, an operation signal is transmitted to the electric cautery apparatus 3 via the foot switch controller 5. In response, the electric cautery apparatus 3 outputs high-frequency energy, which is transmitted to the treating part 7c via a conductive member such as the ultrasonic transmission rod disposed in the sheath 7a and applied to a patient's body part such as tissue nipped by the treating part 7c.

In this situation, a high-frequency current flowing into the body part being treated is collected by an opposite plate 13, which is in broad contact with a patient, and returned to the electric cautery apparatus 3 via a cable 14 connected to the opposite plate 13.

The electric cautery/hemostat 8 also has an elongated sheath 8a, on the back end of which there are provided Handles 8d for controlling the opening/closing of pivoting portions of the treating part 8c on the front end of the sheath 8a are provided on the operating part 8b on the back end of the sheath 8a. The electric cautery/hemostat 8 is connected to the other output port Y of the electric cautery apparatus 3 via a cable 15 removably connected to an electrode 8e formed on the operating part 8b.

When the electric cautery/hemostat 8 is in a standby state achieved by operating the remote controller 9b, if the pedal A or B of the foot switch unit 4 is pressed, an operation signal is transmitted to the electric cautery apparatus 3 via the foot switch controller 5. In response, the electric cautery apparatus 3 outputs high-frequency energy to the treating part 8c on the front end via the conductive member in the sheath 8a. The high-frequency energy is applied to a patient's body part nipped by the treating part 8c thereby performing cutting or coagulating.

FIG. 2 shows the construction of the remote controller 9a (the remote controller 9b also has a similar construction) and the construction of the foot switch controller 5. The remote controller 9a includes an ID generator 17 for generating identification information (referred to as ID) uniquely assigned to the remote controller 9a, a push button switch 18 for selecting the remote controller 9a and more specifically for selecting a mode in which a treating tool assigned to the remote controller 9a is enabled, an oscillator/controller 20 for, in response to an operation of the push button switch 18, outputting an oscillation signal indicating an modulated ID in the form of a radio wave from an antenna 19 to the foot switch controller 5 and also for controlling an LED 21 such that the LED 21 is lit when a selection acknowledgement signal is returned from the foot switch controller 5, the LED 21 that is lit when the remote controller 9a is selected, and a battery 22 for supplying operating power to the oscillator/controller 20.

The foot switch controller 5 includes an antenna 24 for receiving and transmitting a radio wave from or to the remote controller 9a or 9b, a transmitter/receiver 25 connected to the antenna 24, for transmitting a modulated signal in the form of a radio wave to the remote controller 9a or 9b and for receiving and demodulating a signal, an ID discriminator 26 for discriminating an ID from a signal received and demodulated by the transmitter/receiver 25, a foot switch control circuit 27 connected to the transmitter/receiver 25 and also to the ID discriminator 26, for controlling the foot switch unit 4, and a control panel 28 for setting a control function of the foot switch control circuit 27, wherein the foot switch control circuit 27 is connected, via external terminals, to the ultrasonic generator 2, the electric cautery apparatus 3, the foot switch unit 4, and the remote switch unit 6.

As shown in FIG. 3, a clip 29 is connected to the remote controller 9a so that the remote controller 9a can be easily attached (in a removable fashion) to the cable 11 or 12 using the clip 29 thereby disposing the remote controller 9a in the vicinity of a treating tool (scissors hand piece 7, in this specific example) to be used. The remote controller 9a may be attached to the scissors hand piece 7 instead of the cable 11. Similarly, the remote controller 9b can be attached to the cable 15 or the like so that the remote controller 9b is disposed in the vicinity of the electric cautery/hemostat 8.

FIG. 4 shows an example of the construction of the foot switch control circuit 27.

The pedals A and B of the foot switch unit 4 are connected to ports a and b, respectively, of a CPU 31 so that the CPU 31 can detect the operation signals of the pedals A and B of the foot switch unit 4.

A ROM 32, in which information indicating the correspondence between remote controllers and their IDs is stored, is connected to the CPU 31. If an ID discriminated by the ID discriminator 26 is input to the CPU 31, the CPU 31 reads information from the ROM 32 at an address indicated by the ID thereby determining the remote controller corresponding to the ID.

The CPU 31 is also connected to a memory 33 for storing setting data defined by operating a setting key 28a on the control panel 28 so that the CPU 31 controls the operation in accordance with the setting data stored in the memory 33.

More specifically, as will be described later with reference to FIG. 5, setting data indicating the correspondence between remote controllers and treating tools and setting data defining operations that should be performed under the control of the CPU 31 when the pedals A and B of the foot switch unit 4 are pressed are input by operating the setting key 28a, and they are stored in the memory 33, and the CPU 31 performs corresponding control operation by pressing the foot switch unit 4.

As will be described later, in the case where setting is made such that the remote control 9a is assigned to the scissors hand piece 7, if the CPU 31 detects that the push button switch 18 of the remote controller 9a is pushed, the CPU 31 brings the scissors hand piece 7 into a standby state in which the scissors hand piece 7 is ready for use. In this situation, if a treatment start command is issued from the foot switch unit 4, the CPU 31 controls the operation such that ultrasonic energy or high-frequency electric power is supplied to the scissors hand piece 7.

When the remote controller 9a or the 9b is operated, the CPU 31 determines which remote controller 9a or 9b is operated on the basis of the ID and transmits selection acknowledgement information via the transmitter/receiver 25 to inform the controller 9a or 9b that the ID thereof has been detected.

Upon detecting the selection acknowledgement information via the oscillator/controller 20, the remote controller 9a or 9b turns on the LED 21. Thus, if a human operator operates, for example, the remote controller 9a to enable the scissors hand piece 7, the foot switch controller 5 discriminates the ID of the remote controller 9a and returns a selection acknowledgement signal, which causes the remote controller 9a to turn on the LED 21 thereby informing the human operator that the scissors hand piece 7 in connection with the remote controller 9a has been brought into the standby state in which the scissors hand piece 7 is ready for use.

Only when the push button switch 18 is pushed, the remote controller 9a or 9b becomes capable of receiving a selection acknowledgement signal from the foot switch controller 5, and the LED 21 is lit as long as the selection acknowledgement signal is being received. If the transmission of the selection acknowledgement signal is stopped, the LED 21 is turned off to indicate that the treating tool in connection with the remote controller has been disabled.

In this system constructed in the above-described manner, even if acknowledgement signals at the same frequency are transmitted from the transmitter/receiver 25 to both remote controllers 9a and 9b, a remote controller whose push button switch 18 is not pushed is not capable of receiving the acknowledgement signal.

In the case where when one remote controller, for example, 9a, is in a selected state, if the other remote controller 9b is further selected, the CPU 31 stops the transmission of the selection acknowledgement signal for a while in response to detecting the ID of the remote controller 9b thereby disabling the remote controller 9a, and then the CPU 31 transmits a selection acknowledgement signal so as to bring the remote controller 9b into a selected state. In response, the LED of the remote controller 9b is lit. Thus, the human operator can easily determine which remote controller is in a selected state and thus which treating tool is enabled in response to the selection of the remote controller.

As shown in FIG. 4, the CPU 31 is connected to the ultrasonic generator 2 via two ports Ua and Ub and to the electric cautery apparatus 3 via two ports Da and Db.

For example, a signal for controlling the turning-on/off of the ultrasonic power for cutting/coagulating is output from the port Ua; a signal for controlling the output level of the ultrasonic power is output from the port Ub; a signal for controlling the turning-on/off of the high-frequency electric power for cutting is output from the port Da;,and a signal for controlling the turning-on/off of the high-frequency electric power for coagulating is output from the port Db.

In the present embodiment, a mode selection switch 28b for selecting a mixed output mode is disposed on the control panel 28.

In the mixed output mode, if a treatment power output command is issued by pressing the pedal A or B of the foot switch unit 4, high-frequency power and ultrasonic power are successively output in response to only the single command, and thus treatment can be made in a more efficient manner than can be made by individually issuing output commands.

In this mode, the first period of time during which output power is supplied before being switched to second output power can be increased or reduced by operating an up-switch 28c or a down-switch 28a disposed on the control panel 28 thereby changing the setting of a timer 34 provided in the CPU 31.

In the system constructed in the above-described manner according to the present embodiment of the invention, treatment energy can be output to a plurality of treating tools by operating the single foot switch unit 4, wherein a desired treating tool can be enabled by operating a corresponding remote controller 9a or 9b (setting means) that is disposed near the treating tools so that a human operator can easily operate it. Each remote controller 9a or 9b has the LED 21 for indicating whether the remote controller 9a or 9b is in a selected state so that the human operator can easily confirm that a treating tool to be used is in an enabled state.

The operation in accordance with the present embodiment is described below with reference to a flow chart shown in FIG. 5.

Before starting a surgery, the scissors hand piece 7 and the electric cautery/hemostat 8 to be used are connected to the ultrasonic generator 2 and the electric cautery apparatus 3. Furthermore, the foot switch unit 4 is connected to the foot switch controller 5.

The remote controllers 9a and 9b for selecting the scissors hand piece 7 or the electric cautery/hemostat 8 are attached using clips 29 such that they are disposed near the scissors hand piece 7 and the electric cautery/hemostat 8, respectively. After connecting the respective apparatuses/tools as shown in FIG. 1, a presetting process depending on the device connections is performed as shown in FIG. 5.

First, in step S1, the remote controllers 9a and 9b are assigned to the scissors hand piece 7 and the electric cautery/hemostat 8, respectively. That is, by operating the setting key 28a on the control panel 28, a remote controller is assigned to one treating tool connected such that the treating tool can be selected via the remote controller.

Thereafter, in step S2, setting of the remote controller 9a is performed to define what is to be performed when the foot switch unit 4 is operated in a state in which the scissors hand piece 7 is enabled by operating the remote controller 9a.

More specifically, for example, the foot switch controller 27 is set such that the ultrasonic generator 2 outputs (generates) ultrasonic power when the pedal A of the foot switch unit 4 is pressed, and the electric cautery apparatus 3 outputs high-frequency power for coagulation from the port X when the pedal B is pressed.

In the next step S3, setting of the remote controller 9b is performed to define what is to be performed when the foot switch unit 4 is operated in a state in which the electric cautery/hemostat 8 is enabled by operating the remote controller 9b.

More specifically, for example, the foot switch controller 28 is set such that the electric cautery apparatus 3 outputs high-frequency power for cutting from the port Y when the pedal A of the foot switch unit 4 is pressed, and the electric cautery apparatus 3 outputs high-frequency power for coagulation from the port Y when the pedal B is pressed. Thus, the presetting process is completed.

After completion of the presetting process, a human operator pushes the push button switch 18 of the remote controller 9a or 9b disposed near a hand piece to be used. In response to a signal from the operated remote controller, the foot switch controller 5 enables the hand piece corresponding to the operated remote controller.

More specifically, for example, the remote controller 9a. is disposed in the vicinity of the scissors hand piece 7, and the foot switch controller 5 is preset via the control panel 28 such that the remote controller 9a is assigned to the scissors hand piece 7, as shown in FIG. 5.

When the remote controller 9a is operated, a signal is sent from the foot switch controller 5 to the remote controller 9a and the LED 21 of the remote controller 9a is lit in response to receiving the signal so as to indicate that the remote controller 9a is selected.

Thus, the indication of the LED 21 allows the human operator to confirm that the scissors hand piece 7 is selected and is ready for use. After confirmation, if the pedal A or B of the foot switch unit 4 is pressed, ultrasonic power or high-frequency power is output from a corresponding apparatus thereby allowing the human operator to make surgical treatment using the scissors hand piece 7.

Similarly, the electric cautery/hemostat 8 can be enabled by pushing the push button switch 18 of the remote controller 9b. In response, the LED 21 of the remote controller 9b is lit to indicate that the electric cautery/hemostat 8 is selected.

After confirming that the electric cautery/hemostat 8 is selected, if the foot switch unit 4 is operated, the electric cautery apparatus 3 outputs high-frequency power for cutting or coagulating.

As described above, in the system according to the present embodiment, the human operator can easily switch the treating tool as required by operating the wireless remote controller 9a or 9b, and the human operator can easily determine which treating tool is in an enabled state. This allows the human operator to perform surgery in an effective manner.

In the case where the mixed output mode is selected by the mode selection switch 28b, the scissors hand piece 7 can successively output high-frequency cutting power and ultrasonic power (in response to only one operation of the foot switch unit 4) as shown in FIGS. 6A to 6C and FIGS. 7A to 7C thereby allowing mixed treatment to be made.

More specifically, after selecting the mixed output mode by the mode selection switch 28b and making presetting such that the scissors hand piece 7 is selected when the remote controller 9a is operated, if the pedal A is pressed, high-frequency cutting power is first output for a predetermined period of time and then full power (100% power) of ultrasonic vibration is output, as shown in FIGS. 6A to 6C.

On the other hand, if the pedal B is pressed, high-frequency power for coagulation is first output for a predetermined period of time and then middle power (70% power, for example) of ultrasonic vibration is output, as shown in FIGS. 7A to 7C.

This makes it possible to first thermally alter tissue in an instant by applying high-frequency electric power for a short time and then perform cutting or coagulating at a rather low temperature by applying ultrasonic vibration.

The period of time during which the high-frequency electric power is applied can be adjusted using the up-switch 28c or the down-switch 28a.

Thus, the system according to the present embodiment of the invention makes it possible to perform cutting and coagulating in a quick and effective manner using ultrasonic power in conjunction with auxiliary high-frequency energy.

Second Embodiment

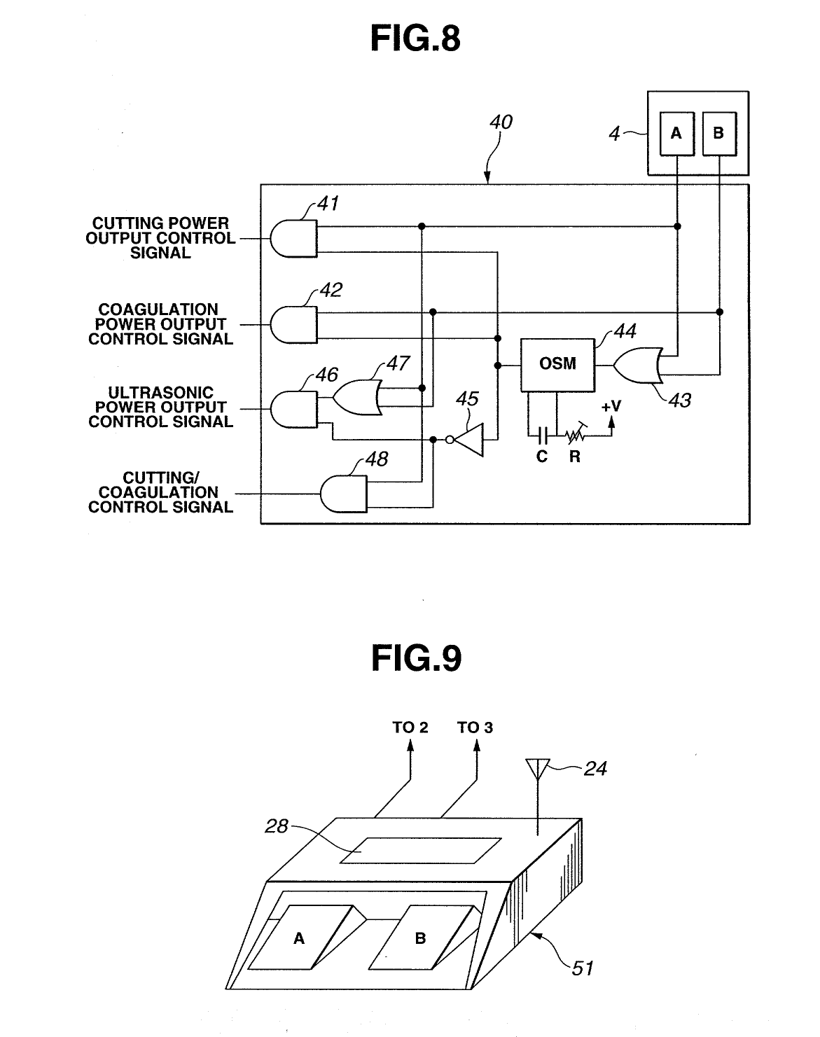

A second embodiment of the present invention is described below with reference to FIG. 8. In this second embodiment, the foot switch controller 27 is constructed in a different fashion from that of the first embodiment described above.

In the foot switch controller 40 according to the present embodiment, as shown in FIG. 8, an on/off-switching signal generated by the pedal A or B of the foot switch unit 4 is supplied as a cutting power output control signal or a coagulation power output control signal to the electric cautery apparatus 3 via an AND gate 41 or 42 and also supplied as a trigger signal to a one-shot multivibrator (OSM) 44 via an OR gate 43.

In response to the rising edge of the trigger signal, the OSM 44 immediately outputs an H-level pulse for a period of time corresponding to a time constant determined by a capacitor C and a resistor R connected to the OSM 44.

The signal output from the OSM 44 is input to the AND gates 41 and 42 and is also output as an ultrasonic power output control signal to the ultrasonic generator 2 via an inverter 45 and an AND gate 46.

To the other input terminal of the AND gate 46, supplied is the on/off-switching signal from the pedal A or B.

The ultrasonic power can be output in a mode suitable for cutting or coagulating in response to a cutting/coagulating control signal that is output to the ultrasonic generator 2 via an AND gate 48 in accordance with, for example, the signal output from the inverter 45 and the on/off-switching signal from the pedal A. When an H-level cutting/coagulating control signal is output, 100% power of ultrasonic power is output, while 70% power of ultrasonic power is output when an L-level cutting/coagulating control signal is output.

The resistance of the resistor R of the OSM 44 can be varied by operating the control panel 28. If the resistance of the resistor R is increased, the first period of time during which the cutting power or coagulating power is output from the electric cautery apparatus 3 increases.

When the pedal A or B is pressed, power is output, as shown in FIGS. 6A to 6C or 7A to 7C, in response to control signals output from the foot switch controller 40.

The present embodiment provides functions and advantages similar to those provided by the first embodiment.

FIG. 9 shows a foot switch unit 51 obtained by disposing the foot switch controller 5 within the foot switch unit 4 according to the first embodiment. The individual parts are similar to those described above except that the foot switch controller 5 is disposed inside the enclosure of the foot switch unit 51.

This makes it unnecessary to provide an additional separate controller, and thus a space created in the operating room by eliminating the need for the separate controller.

In the first embodiment described earlier, communication between the remote controller 9a or 9b and the foot switch controller 5 is performed wirelessly using a radio wave. Alternatively, communication may be performed wirelessly using an ultrasonic wave or an infrared ray.

Having described the preferred embodiments of the invention referring to the accompanying drawings, it should be understood that the present invention is not limited to those precise embodiments, and various changes and modifications thereof could be made by one skilled in the art without departing from the spirit or scope of the invention as defined in the appended claims.

What is claimed is:

1. A surgical system comprising:
a first treating apparatus for generating first treating energy;
a first treating tool for treating a patient's body part on the basis of the first treating energy generated by the first treating apparatus;
a first setting switch disposed in the vicinity of the first treating tool, for making setting such that the first treating energy is used in treatment;
a second treating apparatus for generating second treating energy;
a second treating tool for treating a patient's body part on the basis of the second treating energy generated by the second treating apparatus;
a second setting switch disposed in the vicinity of the second treating tool, for making setting such that the second treating energy is used in treatment;
a display for indicating the states of the first and second setting switches;
a foot switch for issuing a treatment start command; and
a controller for controlling the first and second treating apparatuses in accordance with the states of the first and second setting switches and the foot switch.

2. A surgical system comprising:
a first treating apparatus for generating first treating energy;
a second treating apparatus for generating second treating energy;
first and second treating tools for treating a patient's body part on the basis of the first and second treating energy generated by the first and second treating apparatuses;
first and second setting switches disposed in the vicinity of the first and second treating tools, for selecting the first and second treating energy as treating energy used in the treatment;
a display for indicating the states of the first and second setting switches;
a foot switch for issuing a treatment start command; and
a controller for controlling the first and second treating apparatuses in accordance with the states of the first and second setting switches and the foot switch.

3. A surgical system according to claim 1, wherein the controller transmits and receives a setting signal to or from the first and second setting switches by means of wireless transmission and receives a command signal from the foot switch via a cable.

4. A surgical system according to claim 1, wherein the controller automatically performs a presetting process comprising the steps of assigning the first setting switch to the first treating apparatus; assigning the second setting switch to the second treating apparatus; defining a function of the first setting switch performed in response to an operation of the foot switch; and defining a function of the second setting switch performed in response to an operation of the foot switch.

5. A surgical system according to claim 1, wherein the controller includes a control panel for setting an operation controlled by the foot switch and defining a set value in each operation mode.

6. A surgical system according to claim 2, wherein the controller transmits and receives a setting signal to or from the first and second setting switches by means of wireless transmission and receives a command signal from the foot switch via a cable.

7. A surgical system according to claim 2, wherein the controller automatically performs a presetting process comprising the steps of assigning the first setting switch to the first treating apparatus; assigning the second setting switch to the second treating apparatus; defining a function of the first setting switch performed in response to an operation of the foot switch; and defining a function of the second setting switch performed in response to an operation of the foot switch.

8. A surgical system according to claim 2, wherein the controller includes a control panel for setting an operation controlled by the foot switch and defining a set value in each operation mode.

9. A surgical system comprising:
an ultrasonic generator for generating an ultrasonic energy;
a first treating tool for treating a patient's body part on the basis of the ultrasonic energy generated by the ultrasonic generator;
a first setting switch disposed in the vicinity of the first treating tool, for making setting such that the ultrasonic energy is used in treatment;
a high-frequency electric cautery apparatus for generating high-frequency energy;
a second treating tool for treating a patient's body part on the basis of the high-frequency energy generated by the high-frequency electric cautery apparatus;
a second setting switch disposed in the vicinity of the second treating tool, for making setting such that the high-frequency energy is used in treatment;
a display for indicating the states of the first and second setting switches;
a foot switch for selectively issuing a treatment start command to the ultrasonic generator or the high-frequency electric cautery apparatus; and
a controller for controlling the ultrasonic generator the high-frequency electric cautery apparatus in accordance with the setting made by the first and second setting switches and in response to the treatment start command issued by the foot switch.

10. A surgical system according to claim 9, wherein the controller transmits and receives a setting signal to or from the first and second setting switches by means of wireless transmission and receives a command signal from the foot switch via a cable.

11. A surgical system according to claim 9, wherein the controller automatically performs a presetting process comprising the steps of assigning the first setting switch to the ultrasonic apparatus; assigning the second setting switch to the high-frequency electric cautery apparatus; defining a function of the first setting switch performed in response to an operation of the foot switch; and defining a function of the second setting switch performed in response to an operation of the foot switch.

12. A surgical system according to claim 9, wherein the controller includes a control panel for setting an operation controlled by the foot switch and defining a set value in each operation mode.

13. A surgical system according to claim 1, further comprising third treating means capable of receiving both the first treating energy generated by the first treating apparatus and the second treating energy generated by the second treating apparatus.

14. A surgical system according to claim 13, wherein when the first treating energy generated by the first, treating apparatus and the second treating energy generated by the second treating apparatus are applied to a patient's body part via the third treating means, the controller controls the supply of energy such that the second treating energy is supplied after supplying the first treating energy for a predetermined period of time.

15. A surgical system according to claim 1, wherein the controller controls the second treating apparatus such that the second treating apparatus is maintained in a standby state in which outputting is disabled, when the first treating apparatus is in operation.

16. A surgical system according to claim 13, wherein the controller controls the second treating apparatus such that the second treating apparatus is maintained in a standby state in which outputting is disabled as long as the first treating apparatus is in operation.

17. A surgical system according to claim 1, wherein the first and second setting switches are removably attached to the first and second treating tools, respectively.

18. A surgical system according to claim 1, wherein each of the first and second setting switches is formed in an integral fashion with the display such that a display surface of the display is exposed.

19. A surgical system according to claim 18, wherein the display is a light emitting diode for indicating the setting state of the first or second setting switch.

20. A surgical system according to claim 1, wherein the controller is formed, in an integral fashion, in the inside of the foot switch unit.
